SEQUENCE LISTING (11 sequences)

SEQ ID NO: 1 - DNA, 477 bp, Human papillomavirus
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 2 - DNA, 477 bp, Human papillomavirus
ttacagctgggtttctctacgtgttcttgatgatctgcaacaagacatacatcgaccggtccaccgaccccttatattatggaatctttgctttttgtccagatgtctttgcttttcttcaggacacagtggcttttgacagttaatacacctaattaacaaatcacacaacggtttgttgtattgctgttctaatgttgttccatacaaactataacaataatgtctatactcactaattttagaataaaactttaaacatttatcacatacagcatatggattcccatctctatatactatgcataaatcccgaaaagcaaagtcatatacctcacgtcgcagtaactgttgcttgcagtacacacattctaatattatatcatgtatagttgtttgcagctctgtgcataactgtggtaactttctgggtcgctcctgtgggtcctgaaacattgcagttctcttttggtgcat

SEQ ID NO: 3 - DNA, 20 bp, Human papillomavirus
tgcttttcttcaggacacag

SEQ ID NO: 4 - DNA, 20 bp, Human papillomavirus
tgcagctctgtgcataactg

SEQ ID NO: 5 - RNA, 100 nt, Artificial Sequence
  Guide RNA
ugcuuuucuucaggacacagguuuuagagcuagaaauagcaaguuaaaauaaggcuaguccguuaucaacuugaaaaaguggcaccgagucggugcuuuu

SEQ ID NO: 6 - RNA, 100 nt, Artificial Sequence
  Guide RNA
ugcagcucugugcauaacugguuuuagagcuagaaauagcaaguuaaaauaaggcuaguccguuaucaacuugaaaaaguggcaccgagucggugcuuuu

SEQ ID NO: 7 - DNA, 524 bp, Artificial Sequence
  Designed DNA to act as a target nucleotide to be detected in the Example 1
tcgaagggtgattggatcggagataggatgggtcaatcgtagggacaatcgaagccagaatgcaagggtcaatggtacgcagaatggatggcacttagctagccagttaggatccgactatccaagcgtgtatcgtacggtgtatgcttcggagtaacgatcgcactaagcatggctcaatcctaggctgataggttcgcacatagcatgccacatacgatccgtgattgctagcgtgattcgtaccgagaactcacgccttatgactgcccttatgtcaccgcttatgtctcccgatatcacacccgttatctcagccctaatctctgcggtttagtctggccttaatccatgcctcatagctaccctcataccatcgctcataccttccgacattgcatccgtcattccaaccctgattcctacggtctaacctagcctctatcctacccagttaggttgcctcttagcatccctgttacgtacgctcttaccatgcgtcttaccttggcactatcgatggg

SEQ ID NO: 8 - DNA, 20 bp, Artificial Sequence
  Designed DNA to be detect in the Target nucleotide
agggtcaatggtacgcagaa

SEQ ID NO: 9 - DNA, 20 bp, Artificial Sequence
  Designed DNA to be detect in the Target nucleotide
cattccaaccctgattccta

SEQ ID NO: 10 - RNA, 100 nt, Artificial Sequence
  Guide RNA
agggucaaugguacgcagaaguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence
  Guide RNA
cauuccaacccugauuccuaguuuuagagcuagaaauagcaaguuaaaauaaggcuaguccguuaucaacuugaaaaaguggcaccgagucggugcuuuu

(19) United States
(12) Patent Application Publication
(10) Pub. No.: US 2022/0090174 A1
(43) Pub. Date: Mar. 24, 2022

(54) DETECTION METHOD FOR A TARGET NUCLEIC ACID AND KIT

(71) Applicants: CANON KABUSHKI KAISHA, TOKYO (JP); CANON MEDICAL SYSTEMS CORPORATION, TOCHIGI (JP)

(72) Inventors: Mie Okano, Kanagawa (JP); Yoji Yamamoto, Tokyo (JP); Masato Minami, Kanagawa (JP); Tetsuya Yano, Ibaraki (JP)

(21) Appl. No.: 17/469,506

(22) Filed: Sep. 8, 2021

(30) Foreign Application Priority Data
Sep. 14, 2020 (JP) 2020-154144

(51) Int. Cl. C12Q 1/6818; C12N 15/11; C12N 9/22

(52) U.S. Cl. CPC C12Q 1/6818; C12N 2310/20; C12N 9/22; C12N 15/111

(57) ABSTRACT

A detection method for a target nucleic acid comprising: a first step of contacting a target nucleic acid with a first guide RNA and a first Cas protein; a second step of contacting the target nucleic acid with a second guide RNA and a second Cas protein; and a third step of detecting a complex comprising the target nucleic acid, the first guide RNA, the first Cas protein, the second guid RNA, and the second Cas protein, wherein, in the complex, the first guide RNA and the first Cas protein are bound to the first base sequence, and the second guide RNA and the second Cas protein are bound to the second base sequence.

### BACKGROUND OF THE INVENTION

### Field of the Invention

The present invention relates to a detection method for a target nucleic acid and kit.

### Description of the Related Art

Nucleic acid is the material that carries the genetic information of all organisms, including viruses, and is detected or quantified in various fields. In particular, in the medical field, techniques for the detection and quantification of nucleic acids are used for the detection of pathogens such as viruses and bacteria and genetic tests related to lesions, and there is an increasing demand for more rapid, accurate, and versatile techniques for the detection or quantification of nucleic acids.

In recent years, the CRISPR-Cas system in bacteria and archaebacteria has been discovered, and Japanese Patent Application Laid-Open No. 2017-530695 proposes a detection method for a nucleic acid which can be carried out quickly under a wide range of working conditions using the CRISPR-Cas system.

### SUMMARY OF THE INVENTION

In the CRISPR-Cas system, guide RNA and Cas proteins recognize and bind to sequences of portions of the target nucleic acid. However, guide RNA and Cas proteins can, in rare case, bind to other nucleic acids having the same sequences as the sequence of a part of the target nucleic acids or other nucleic acids having sequences similar to the sequences of the target nucleic acids. In other words, in the CRISPR-Cas system, so-called off-target is known to occur in rare case. Therefore, in the technique described in Japanese Patent Application Laid-Open No. 2017-530695, when a nucleic acid having a different sequence which is the same as the sequence of a part of the target nucleic acid or very similar to the sequence of the target nucleic acid exists, there is a possibility that a false positive reaction occurs due to the off-target, and there is room for improving the specificity.

Accordingly, an object of the present invention is to provide a detection method for a target nucleic acid that is rapid, versatile and has even higher specificity, and a kit for carrying out the detection method for the target nucleic acid.

A detection method for a target nucleic acid according to one aspect of the present invention comprises:

a first step of contacting a target nucleic acid with a first guide RNA and a first Cas protein, the first guide RNA recognizing a first base sequence of the target nucleic acid;

a second step of contacting the target nucleic acid with a second guide RNA and a second Cas protein, the second guide RNA recognizing a second base sequence of the target nucleic acid and the second base sequence being different from the first base sequence;

a third step of detecting a complex comprising the target nucleic acid, the first guide RNA, the first Cas protein, the second guide RNA and the second Cas protein, wherein the first guide RNA and the first Cas protein are bound to the first base sequence and the second guide RNA and the second Cas protein are bound to the second base sequence in the complex.

A kit according to an another aspect of the present invention is a kit for detecting a target nucleic acid, the kit comprising a first guide RNA, a first Cas protein, a second guide RNA, and a second Cas protein, wherein the first guide RNA has a sequence capable of recognizing a first base sequence of the target nucleic acid, the second guide RNA has a sequence capable of recognizing a second base sequence of the target nucleic acid, and the second base sequence is different from the first base sequence.

Further features of the present invention will become apparent from the following description of exemplary embodiments with reference to the attached drawings.

### BRIEF DESCRIPTION OF THE DRAWINGS

FIG. 1 is a schematic diagram of a complex detected by a detection method for a target nucleic acid according to the present invention.

FIGS. 2A, 2B, 2C and 2D are schematic diagrams illustrating an example of immobilizing the Cas protein on a substrate and detecting the complex by a signal generated by using a labeling substance.

FIGS. 3A, 3B and 3C are schematic diagrams illustrating detection of a target nucleic acid utilizing aggregation of latex particles.

FIGS. 4A, 4B and 4C are schematic diagrams illustrating detection of a target nucleic acid using a lateral flow assay.

FIGS. 5A, 5B, 5C, 5D and 5E are schematic diagrams illustrating steps of a method to detect a target nucleic acid by immobilizing Cas protein on a substrate.

FIGS. 6A and 6B illustrate the relationship between the concentration of the target nucleic acid and the absorbance obtained by measurement.

### DESCRIPTION OF THE EMBODIMENTS

Embodiments for carrying out the present invention will be described below. It should be noted that the present invention is defined by the claims, and is not limited to the following embodiments and examples. For example, the materials, composition conditions, reaction conditions, and the like of the following embodiments and examples can be freely modified to the extent understood by those skilled in the art to realize the present invention.

A detection method for a target nucleic acid according to the present invention comprises:

a first step of contacting a target nucleic acid with a first guide RNA and a first Cas protein, the first guide RNA recognizing a first base sequence of the target nucleic acid;

a second step of contacting the target nucleic acid with a second guide RNA and a second Cas protein, the second guide RNA recognizing a second base sequence of the target nucleic acid and the second base sequence being different from the first base sequence; and

a third step of detecting a complex comprising the target nucleic acid, the first guide RNA, the first Cas protein, the second guide RNA and the second Cas protein, wherein the first guide RNA and the first Cas protein are bound to the first base sequence and the second guide RNA and the second Cas protein are bound to the second base sequence in the complex.

(Target Nucleic Acid)

The term “target nucleic acid ” as used herein refers to a nucleic acid to be detected, and includes, for example, deoxyribonucleic acid (DNA), ribonucleic acid (RNA), and the like. The “target nucleic acid sequence ” refers to a base sequence of a target nucleic acid mainly composed of adenosine (A), cytosine (C), guanine (G), thymine (T) and uracil (U). The target nucleic acid may be single-stranded or double-stranded.

The target nucleic acid is, for example, derived from a microorganism, and in some embodiments, the target nucleic acid is derived from a microorganism having a pathogenicity. Here, the microorganisms include, for example, viruses, bacteria and fungi. The virus includes a DNA virus having DNA as a genome and an RNA virus having RNA as a genome.

The specimen in the present invention is an object to be tested for the presence or absence of a target nucleic acid, the amount (concentration),the ratio of a detected amount (concentration) to a reference value of the amount (concentration), and the like, and may contain only the target nucleic acid, or may contain a mixture of a plurality of nucleic acids. Depending on the concentration of the target nucleic acid in the specimen, a known nucleic acid amplification technique such as an isothermal amplification method or a variable temperature amplification method, for example, a PCR method, a LAMP method, a RT-LAMP method and a NASBA method may be applied in advance to amplify the target nucleic acid, and then the target nucleic acid may be subjected to the detection method according to the present invention. In the case of a sample containing contaminants such as blood, saliva and living things, the sample may be used for detection as it is, or may be used for detection after being subjected to purification using a known technique or a commercially available nucleic acid extraction kit.

The concentration of the target nucleic acid in the specimen is preferably in the range of 0 to 100 nM.

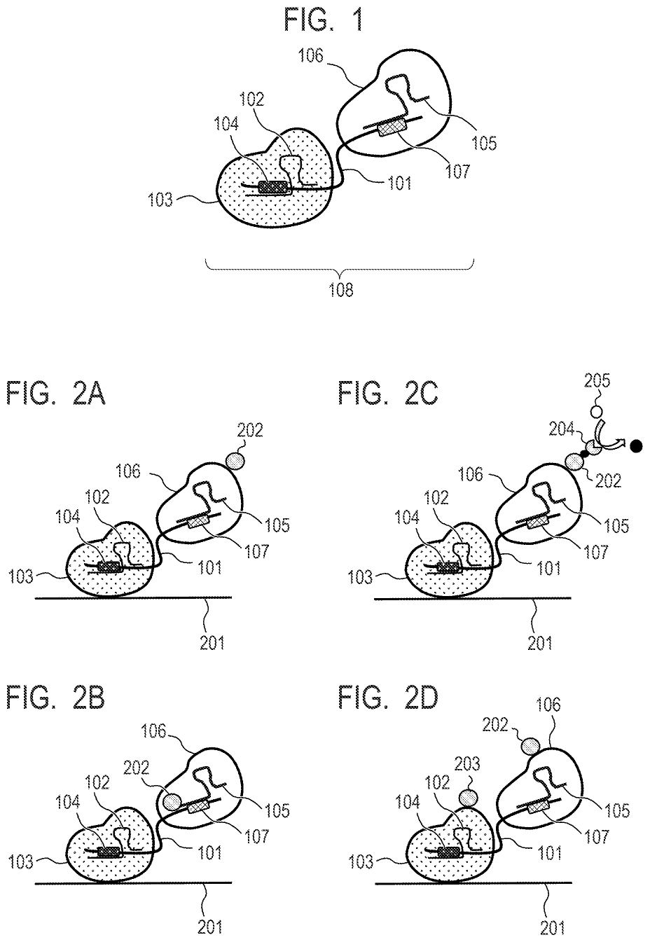

Each step in the target nucleic acid detection method according to the present invention will be described below with reference to FIG. 1. FIG. 1 is a schematic diagram of a complex detected by a detection method for a target nucleic acid according to the present invention.

(First Step)

In a first step, the target nucleic acid 101 is contacted with the first guide RNA 102 and the first Cas protein 103. The first guide RNA 102 recognizes a first base sequence 104 of the target nucleic acid 101, and the first guide RNA 102 and the first Cas protein 103 bind to the target nucleic acid 101.

The term “guide RNA” in the CRISPR-Cas system is known and is utilized for the detection of a target nucleic acid and is responsible for recognizing a portion of the sequence possessed by the target nucleic acid. A guide RNA contain a sequence for recognizing a portion of the sequence carried by the target nucleic acid and are responsible for the specificity of detection of the target nucleic acid in the CRISPR-Cas system. The guide RNA must be designed to match the Cas protein described below. Methods for designing and producing a guide RNA are well known, and design tools are provided, as well as guide RNAs are provided and sold by suppliers. The first guide RNA and the second guide RNA described below in the present invention can be prepared in accordance with generally known methods for preparing the guide RNA. In some embodiments, the first guide RNA and the second guide RNA, described below, each comprise from about 20 to about 100 bases.

The first Cas protein and the second Cas protein described below in the present invention both correspond to the term “Cas protein” known in the CRISPR-Cas system.

In the present invention, the Cas protein is capable of binding to a specific site of a target nucleic acid without cleaving the target nucleic acid, depending on the specificity of the guide RNA. For example, the Cas protein may be modified to inactivate nuclease activity. Such modifications include changes in one or more amino acids that inactivate nuclease activity or nuclease domains. Such modifications also include removing one or more polypeptide sequences exhibiting nuclease activity, i.e., the nuclease domain, so that the nuclease domain are not present in the Cas protein.

In one aspect, the target nucleic acid is double-stranded DNA, and the first Cas protein and the second Cas protein are nuclease-deficient Cas9 proteins (dCas9), respectively. Methods of preparing dCas9 are known and commercially available from a number of sources.

Any protein other than dCas9 that can recognize the target nucleic acid and detect the complex without cleavage the target nucleic acid can be used as a first Cas protein or a second Cas protein. For example, nuclease-deficient Cas13b (dCas13b) or RCas9 composed of dCas9, a fluorescent protein and PAMmer or the like can be used as a first Cas protein or a second Cas protein.

Since the Cas protein is known to be selective for the target nucleic acid, it is preferable to select the optimum type of Cas protein from the viewpoint of its selectivity and sensitivity. For example, if double-stranded DNA is to be detected as a target nucleic acid, dCas9 can be used. If RNA is to be detected, a Cas protein that recognizes RNA, such as RCas9 or dCas13b, may be used, or a DNA obtained by reverse transcription of a target RNA may be used as a target nucleic acid and a Cas protein that recognizes DNA may be used for detection.

For example, when Cas13b is used, Prevotella sp. P5-125 (PSp) Cas13b may be used, or Porphyromonas gulae (Pgu) Cas13b may be used.

In the first step, the first guide RNA and the first Cas protein are preferably brought into contact with the target nucleic acid after the first guide RNA and the first Cas protein are mixed under optimal conditions. The first guide RNA, the first Cas protein, and the target nucleic acid may be mixed simultaneously to bring the first guide RNA and the first Cas protein into contact with the target nucleic acid.

(Second Step)

In a second step, the target nucleic acid 101 is contacted with the second guide RNA 105 and the second Cas protein 106. The second guide RNA 105 recognizes the second base sequence 107 of the target nucleic acid 101, and the second guide RNA 105 and the second Cas protein 106 bind to the target nucleic acid 101. The second base sequence 107 is different from the first base sequence 104.

In the second step, the second guide RNA and the second Cas protein are preferably brought into contact with the target nucleic acid after the second guide RNA and the second Cas protein are mixed under optimal conditions. Alternatively, the second guide RNA, the second Cas protein, and the target nucleic acid may be mixed simultaneously to bring the second guide RNA and the second Cas protein into contact with the target nucleic acid.

When the target nucleic acid is double-stranded, the first base sequence and the second base sequence may be on the same strand or on different strands.

Since the guide RNA binds to a sequence part adjacent to the protospacer adjacent motif (PAM) sequence, the first base sequence or the second base sequence is preferably a site adjacent to the PAM sequence. Note that the PAM sequence is unique to each type of Cas protein in bacteria. For example, in the case of the Cas9 protein from Streptococcus pyogenes (spCas9), the PAM sequence is 3 bases (5′-NGG-3′) and the guide RNA binds to the complementary strand of the sequence adjacent to the PAM sequence. When PAMmer which was presented in Nature by Jennifer A. Doudna et al. in UC Berkeley is utilised, the first or second base sequence need not be a site adjacent to the PAM sequence.

The first base sequence and the second base sequence are preferably separated by a certain distance or more in order to avoid steric hindrance between the Cas proteins bonded to the first base sequence and the second base sequence, respectively. Specifically, the target nucleic acid preferably has 20 or more bases between the first base sequence and the second base sequence. More preferably, the target nucleic acid has 100 or more bases between the first base sequence and the second base sequence. Further preferably, the target nucleic acid has 200 or more bases between the first base sequence and the second base sequence. The target nucleic acid preferably has 500 or less bases between the first base sequence and the second base sequence.

As mentioned above, the invention also includes embodiments in which the target nucleic acid is double-stranded and the first base sequence and the second base sequence are on different strands. In this case, the target nucleic acid having 20, 100 or 200 or more bases between the first base sequence and the second base sequence means having 20, 100 or 200 or more base pairs between the first base sequence and the second base sequence, respectively.

As the first base sequence and the second base sequence, for example, when the target nucleic acid is DNA or RNA derived from a virus, a part of the sequence can be selected from conserved regions having little gene mutation. When the type of the virus in which the polymorphism exists is to be distinguished, it is preferable to determine at least one of the first base sequence and the second base sequence by selecting a part of the sequence from a region containing a sequence specific to the type to be detected.

For example, when the target nucleic acid is DNA derived from a pathogenic bacterium, the first base sequence or the second base sequence may be obtained by selecting a part of the sequence from a region containing a gene (pathogenic gene) involved in a disease.

The first step and the second step may be performed either sequentially or simultaneously as long as the composite 108 shown in FIG. 1 is formed.

Alternatively, for example, in the first step, after the first Cas protein and the first guide RNA are contacted with each other, the complex of them may be immobilized to a substrate and the target nucleic acid may be added thereto and then, the second step may be performed. Alternatively, after immobilizing the first Cas protein on the substrate, the first guide RNA and target nucleic acid may be contacted with the immobilized first Cas protein on the substrate, and then the second step may be performed.

Alternatively, after the first step, a complex formed by binding the first Cas protein and the first guide RNA to the target nucleic acid may be immobilized to the substrate, and then the second step may be performed. The immobilization of the Cas protein is not limited thereto, but may be carried out by physical adsorption or by forming a covalent bond by utilizing a functional group possessed by the Cas protein. Alternatively, the Cas protein may be modified to contain, for example, a mercapto group, an amino group, an aldehyde group, a carboxyl group, biotin or the like to impart a functional group to the Cas protein, and then immobilized to a substrate or the like by utilizing the imparted functional group.

(Third Step)

In a third step, a target nucleic acid is detected by detecting a complex 108 comprising a target nucleic acid 101, a first guide RNA 102, a first Cas protein 103, a second guide RNA 105 and a second Cas protein 106. In the complex 108, the first guide RNA 102 and the first Cas protein 103 are bound to the first base sequence 104, and the second guide RNA 105 and the second Cas protein 106 are bound to the second base sequence 107.

In the present invention, two points of the first base sequence and the second base sequence of the target nucleic acid are independently recognized by the first guide RNA and the second guide RNA, and detection is performed. Therefore, the target nucleic acid can be detected with higher specificity than when only one of the first base sequence and the second base sequence is recognized and detected.

That is, in detection, it is contemplated that either the first guide RNA and the first Cas protein set or the second guide RNA and the second Cas protein set non-specifically bind to nucleic acids other than the target nucleic acid to form non-specific aggregates. The formation of non-specific aggregates is due to a phenomenon so-called off-target in the CRISPR-Cas system. However, in the present invention, the probaility for both of the two sequences in the target nucleic acid which are the detetion targets to cause the off-target simultaneiously is very low, and accordingly, the possibility of the false detection is very low.

In one aspect, the detection of the complex in the third step is based on changes in structure and physical properties.

For example, in the case of using a nanopore method, it is possible to detect the complex by observing the change of current when the complex bound to the nucleic acid is moving in the nanopore sensor or when the complex is in proximity to the nanopore or the nanogap sensor. In the present invention, when the complex to be detected invades the nanopore, ion current is reduced because ion flow in the pore is disturbed. When the guide RNA and Cas protein are bound to the target nucleic acid, their entry into the pore further reduces ion flow and reduces ion current.

When the binding of the guide RNA and the Cas protein has occurred in only one site, the decrease in ionic current occurs only once, whereas the binding of the guide RNA and the Cas protein has occurred in two sites, the decrease in ionic current occurs twice. For example, in the detection, a case in which a set of the first guide RNA and the first Cas protein or a set of the second guide RNA and the second Cas protein binds non-specifically to a nucleic acid other than the target nucleic acid to form a non-specific aggregate is considered. At this time, when the target nucleic acid is detected, the ion current is decreased twice, whereas when the non-specific aggregate is detected, the ion current is decreased once. Thus, the complex to be detected can be detected separately from the non-specific aggregate.

For the detection of the complex, a known detection method for a substance in which a nucleic acid and a protein are bound to each other can be used. Examples of this technique include electron microscopes, optical microscopes, scanning probe microscopes, atomic force microscopes, cantilever detection methods, quartz oscillator detection methods, field effect transistors, surface plasmon resonance spectroscopy, depolarization methods, aggregation methods, and the like. In these methods, the differences in the size and the molecular weight between the complex to be detected and the non-specific aggregate are utilized, and they can be detected separately.

In some embodiments, either the second guide RNA or the second Cas protein is associated with the second labeling substance, and in a third step the complex is detected by a second signal generated by using the second labeling substance.

Also, in some embodiments, either the second guide RNA or the second Cas protein is associated with the second labeling substance, and either the first guide RNA or the first Cas protein is associated with the first labeling substance. The first labeling substance is a substance different from the second labeling substance, and in the third step, the complex is detected by two ways of a detection by the first signal generated by using the first labeling substance and a detection by the second signal. For example, fluorescence resonance energy transfer (FRET) may be utilized, such as binding cyan fluorescent protein (CFP) to a first guide RNA and binding yellow fluorescent protein (YFP) to a second guide RNA. By detecting the complex with using two signals of the first signal and the second signal, the complex to be detected can be detected separately from the non-specific aggregate.

For the purpose of increasing the signal intensity for detection or the like, the first labeling substance and the second labeling substance may be the same substance.

As the guide RNA and the Cas protein bound to the labeling substance, a commercially available guide RNA and the Cas protein modified with a tag beforehand may be used, or guide RNA and the Cas protein bound to the labeling substance may be produced and used.

The first and second signals are preferably generated by using at least one selected from the group consisting of a radioactive material, an enzyme, a capture molecule, a fluorescent material, a luminescent material, a metal particle, a protein-protein binding pair, and a protein-antibody binding pair.

As used herein, an antibody includes any class of antibody molecules and fragments thereof, e.g., Fab region fragments.

As the fluorescent substance, the following substances can be used, but are not limited thereto. Yellow fluorescent protein (YFP), green fluorescent protein (GFP), cyan fluorescent protein (CFP), fluorescein, fluorescein isothiocyanate, rhodamine, cyan, Cy3, Cy5, Alexa 568, Alexa 647, and the like.

The luminescent material includes, but is not limited to, luciferase (for example, the one from bacteria, fireflies and click beetles), luciferin and aequorin.

Examples of enzymes used for signaling include, but are not limited to, galactosidase, glucuronidase, phosphatase, peroxidase, cholinesterase, and the like. When an enzyme is used, a visually detectable signal can be obtained.

The radioactive material includes, for example, 125I, 35S, 14C, or 3H.

The protein-protein binding pair includes biotin-enzyme-labeled avidin and the like, and the protein-antibody binding pair includes biotin-fluorescent-labeled anti-biotin antibody and the like.

The materials necessary for systems utilizing labeling substances for generating the signals mentioned above are commercially available from a variety of sources and can be utilized by, for example, applying an labeling substance to a guide RNA or a Cas protein using known techniques. For example, some commercially available Cas proteins already have an affinity tag attached to them, and the tag can be used to attach a labeling substance. For example, dCas9 provided by NEW ENGLAND BIOLABS contains a His tag, which can be used to impart a fluorescent substance or biotin to dCas9.

As the detection methods of the first signal and the second signal, a fluorescence detection method, an electroluminance detection method, a chemiluminescence detection method, a bioluminescence detection method, a colorimetric detection method and the like can be used.

In one aspect, the third step includes obtaining information about the concentration of the target nucleic acid based on the second signal. Information regarding the concentration of the target nucleic acid may also be obtained based on two signals of the first signal and the second signal. The information on the concentration of the target nucleic acid is preferably at least one selected from the group consisting of the presence or absence of the target nucleic acid, the value of the concentration of the target nucleic acid, and the ratio of the concentration of the target nucleic acid to a reference value of the concentration of the target nucleic acid.

In some embodiments, the first Cas protein is immobilized to the substrate and the complex is detected on the solid phase surface. Also, in some embodiments, in addition to the first Cas protein, a second Cas protein is immobilized on the substrate and the complex is detected on the solid phase surface.

As the substrate, as long as the Cas protein or the complex including the Cas protein can be suitably carried, a general substrate such as resin, glass, inorganic material such as silicon, metal, metal oxide, and the like. can be used without being limited in shape, material, and the like.

When light transmission is used for detection, it is preferable to use a glass substrate, a quartz substrate, a resin substrate such as polycarbonate or polystyrene, an ITO substrate or the like which is optically transparent to the wavelength of the incident light and the light to be detected.

In order to covalently fix the Cas protein, a functional group such as an amino group or a carboxyl group may be modified on the surface of the substrate. The shape of the substrate may be flat, such as a plate, or spherical, such as magnetic beads, gold fine particles, or polystyrene beads.

An example in which the first Cas protein is immobilized on a substrate and the complex is detected by a signal generated by using a labeling substance is shown in FIGS. 2A to 2D.

In some embodiments, as shown in FIG. 2A, the first Cas protein 103 is immobilized on the substrate 201, and the complex thereby immobilized on the substrate 201 is detected with a second labeling substance 202 attached to the second Cas protein 106 as a label. Here, the second labeling substance 202 may be attached to the second guide RNA 105 as shown in FIG. 2B. Further, as shown in FIG. 2C, the second labeling substance 202 may be further modified with a catalyst 204 such as an enzyme, and a signal transmitter 205 for transmitting a signal for detection may act on the catalyst 204 to perform detection.

Also, in some embodiments, as shown in FIG. 2D, the first Cas protein 103 immobilized on the substrate 201 is further associated with the first labeling substance 203 and the second Cas protein 106 is associated with the second labeling substance 202. In the detection, detection is performed by two methods: detection using the first labeling substance 203 as a label and detection using the second labeling substance 202 as a label.

When the first Cas protein and the second Cas protein are immobilized on latex particles such as polystyrene beads, the presence or absence of the target nucleic acid can be confirmed by visually detecting the complex by utilizing aggregation of the latex particles. In the detection of the complex by the aggregation method, confirmation of the presence or absence of the target nucleic acid and measurement of the concentration of the target nucleic acid can be performed by measuring the change in absorbance. The complex can also be detected using a lateral flow assay, for example, by immobilizing the first Cas protein on the gold microparticles.

(Kit for Detecting Target Nucleic Acid)

The kit according to the present invention is a kit for detecting a target nucleic acid comprising a first guide RNA, a first Cas protein, a second guide RNA and a second Cas protein, wherein the first guide RNA has a sequence capable of recognizing a first base sequence possessed by the target nucleic acid, the second guide RNA has a sequence capable of recognizing a second base sequence possessed by the target nucleic acid, and the second base sequence is different from the first base sequence.

In some embodiments, the kit further comprises a lateral flow strip.

Also, in some embodiments, the kit further comprises latex particles.

### EXAMPLE

Hereinafter, the present invention will be described in detail with reference to examples, but the present invention is not limited to these examples.

### Example 1

An example utilizing aggregation of latex particles for detection of human papillomavirus (HPV) type 16 is described.

HPV is a member of the papillomavirus family, and it is known that most cervical cancers are caused by HPV infection. There are more than 150 types of HPV, and some types are thought to cause cervical cancer; about 65% of cervical cancers are caused by HPV 16 and HPV 18. HPV 16 and HPV 18 are both high-risk types, but they differ in the degree of risk of developing cancer and the type of cancer they are likely to develop. Therefore, it is important to distinguish HPV 16 and HPV 18 from each other, but HPV 16 and HPV 18 have high DNA sequence homology, and it is considered difficult to detect them based on the DNA sequence.

In this example, the first base sequence and the second base sequence were selected and designed from the antisense strand sequences of the E6 gene of HPV 16. The sequences of the sense strand of the E6 gene, the antisense strand of the E6 gene, the first base sequence, the second base sequence, the first guide RNA and the second guide RNA are shown below, respectively. Here, the specificity of the first guide RNA is somewhat low, and it may bind non-specifically to DNA of HPV 16 as well as DNA of HPV 18 as the target nucleic acid. On the other hand, the second guide RNA is highly specific and binds to DNA of HPV 16 as the target nucleic acid but not DNA of HPV 18. The DNA of the HPV 16 serving as the target nucleic acid has 2 strands, and dCas9 is used as the first Cas protein and the second Cas protein, respectively.

Sense strand of E6 gene of HPV 16
(SEQ ID NO: 1)
ATGCACCAAAAGAGAACTGCAATGTTTCAGGACCCACAGGAGC
GACCCAGAAAGTTACCACAGTTATGCACAGAGCTGCAAACAACTATACA
TGATATAATATTAGAATGTGTGTACTGCAAGCAACAGTTACTGCGACGT
GAGGTATATGACTTTGCTTTTCGGGATTTATGCATAGTATATAGAGATG
GGAATCCATATGCTGTATGTGATAAATGTTTAAAGTTTTATTCTAAAAT
TAGTGAGTATAGACATTATTGTTATAGTTTGTATGGAACAACATTAGAA
CAGCAATACAACAAACCGTTGTGTGATTTGTTAATTAGGTGTATTAACT
GTCAAAAGCCACTGTGTCCTGAAGAAAAGCAAAGACATCTGGACAAAAA
GCAAAGATTCCATAATATAAGGGGTCGGTGGACCGGTCGATGTATGTCT
TGTTGCAGATCATCAAGAACACGTAGAGAAACCCAGCTGTAA
Antisense strand of E6 gene of HPV 16
(SEQ ID NO: 2)
TTACAGCTGGGTTTCTCTACGTGTTCTTGATGATCTGCAACAAGA
CATACATCGACCGGTCCACCGACCCCTTATATTATGGAATCTTTGCTTT
TTGTCCAGATGTCTTTGCTTTTCTTCAGGACACAGTGGCTTTTGACAGT
TAATACACCTAATTAACAAATCACACAACGGTTTGTTGTATTGCTGTTC
TAATGTTGTTCCATACAAACTATAACAATAATGTCTATACTCACTAATT
TTAGAATAAAACTTTAAACATTTATCACATACAGCATATGGATTCCCAT
CTCTATATACTATGCATAAATCCCGAAAAGCAAAGTCATATACCTCACG
TCGCAGTAACTGTTGCTTGCAGTACACACATTCTAATATTATATCATGT
ATAGTTGTTTGCAGCTCTGTGCATAACTGTGGTAACTTTCTGGGTCGCT
CCTGTGGGTCCTGAAACATTGCAGTTCTCTTTTGGTGCAT
First and second base sequences targeted in
detecting HPV 16
First base sequence
(SEQ ID NO: 3)
TGCTTTTCTTCAGGACACAG
Second base sequence
(SEQ ID NO: 4)
TGCAGCTCTGTGCATAACTG
First guide RNA for detecting the first base
sequence of HPV type 16
(SEQ ID NO: 5)
GCUUUUCUUCAGGACACAGGUUUUAGAGCUAGAAAUAGCAA
GUUAAAAUAAGGCUAGUCCGUUAUCAACUUGAAAAAGUGGCACCGAGUC
GGUGCUUUU
A second guide RNA for detecting a second base
sequence of HPV type 16
(SEQ ID NO: 6)
UGCAGCUCUGUGCAUAACUGGUUUUAGAGCUAGAAAUAGCAA
GUUAAAAUAAGGCUAGUCCGUUAUCAACUUGAAAAAGUGGCACCGAGUC
GGUGCUUUU

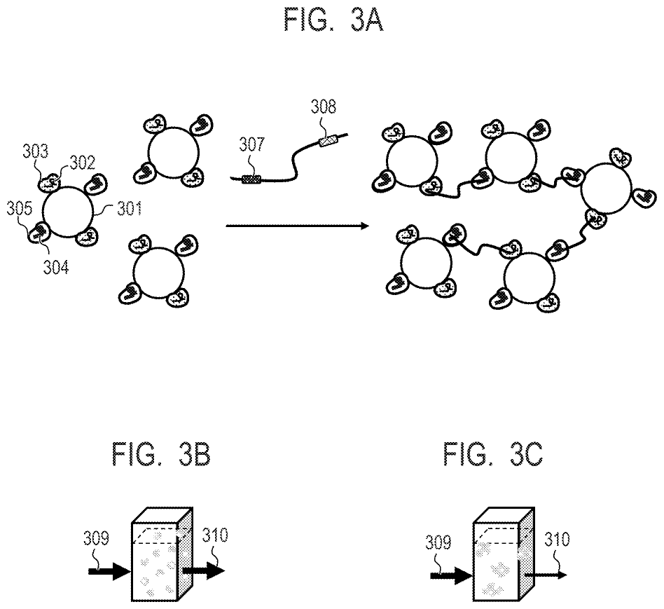

FIG. 3A is a schematic diagram illustrating detection of a target nucleic acid using aggregation of latex particles.

First, as a preliminary preparation, a first guide RNA 302-first Cas protein 303 complex and a second guide RNA 304-second Cas protein 305 complex are formed in advance. They are then immobilized to latex particles 301 to prepare latex particles for target nucleic acid detection. The guide RNA-Cas protein complex can be formed by mixing the guide RNA and the Cas protein and reacting them at 37° C. for 30 minutes. As a method for immobilizing the guide RNA-Cas protein complex to the latex particles, a known method for immobilizing the protein to the latex particles, such as a physical adsorption method or a chemical bonding method, can be used.

Here, a method of immobilizing the guide RNA-Cas protein complex on polystyrene particles used as latex particles 301 using a physical adsorption method is described.

A first guide RNA-first Cas protein complex and a second guide RNA-second Cas protein complex are added to polystyrene particles suspended in phosphate buffered saline (PBS, pH 7.4) and stirred at room temperature for 1 hour. The particles were then washed by centrifugation, subjected to blocking with bovine serum albumin (BSA), washed, and then adjusted to a particle concentration of 0.5 mass % with PBS. Thus, latex particles for detection are prepared by physically adsorbing the first guide RNA-first Cas protein complex and the second guide RNA-second Cas protein complex to polystyrene particles. Although BSA is used as the blocking agent in this embodiment, other blocking agents also can be used instead.

In detection, for example, a sample containing the target nucleic acid 306 is mixed with a solution in which the prepared latex particles for detection are dispersed, and as shown in FIG. 3A, the latex particles for detection are reacted with the target nucleic acid 306 to induce aggregation. As the specimen, it is preferable to extract the double-stranded DNA from a specimen such as cervical mucus using a commercially available kit beforehand.

The induced aggregation can be detected by measuring the absorbance as follows. That is, as shown in FIG. 3B, the absorbance of a solution in which latex particles for detection are dispersed is measured in advance before the sample is mixed. At this time, since the latex particles for detection are substantially uniformly dispersed, the difference between the intensity of the incident light 309 of the specific wavelength and the intensity of the transmitted light 310 is small. Next, as shown in FIG. 3C, the absorbance of a solution in which latex particles for detection are dispersed after mixing the sample is measured. When aggregation is induced, the induced aggregation prevents light transmission, and the difference between the intensity of the incident light 309 and the intensity of the transmitted light 310 becomes large. The degree of aggregation can be assessed from the difference in absorbance measured before and after mixing the samples. Since the degree of aggregation increases as the specimen contains more target nucleic acids, the amount of target nucleic acids contained in the specimen can be evaluated by evaluating the degree of aggregation. Instead of measuring the absorbance, the presence or absence of aggregation may be visually determined. That is, the presence of the target nucleic acid in the specimen can be detected by confirming that aggregation is induced.

Examples of detection by measuring absorbance are described here.

First, 230 μL of PBS as a reaction buffer and 40 μL of a solution in which latex particles for detection prepared above are dispersed are dispensed into a cuvette for measuring absorbance. Next, the absorbance of each dispensed solution at a wavelength of 950 nm is measured. Thereafter, 30 μL of the sample solution containing the target nucleic acid is further added to the cuvette, and the absorbance at a wavelength of 950 nm is measured again. Determine the change in absorbance before and after addition of the test sample, and evaluate the degree of aggregation.

When the target nucleic acid is present in the specimen, as shown in FIG. 3A, the first guide RNA 302-the first Cas protein 303 complex is bound to the first base sequence 307 of the target nucleic acid 306. In addition, the second guide RNA 304-second Cas protein 305 complex is bound to the second base sequence 308. As a result, agglomeration of latex particles is induced, and the absorbance changes accordingly, so that HPV 16 can be detected.

### Example 2

An example utilizing a lateral flow assay for the detection of human papillomavirus (HPV) type 16 is described. Specimens containing the first guide RNA, the first Cas protein, the second guide RNA, the second Cas protein and the target nucleic acid used in this example are all the same as those used in Example 1.

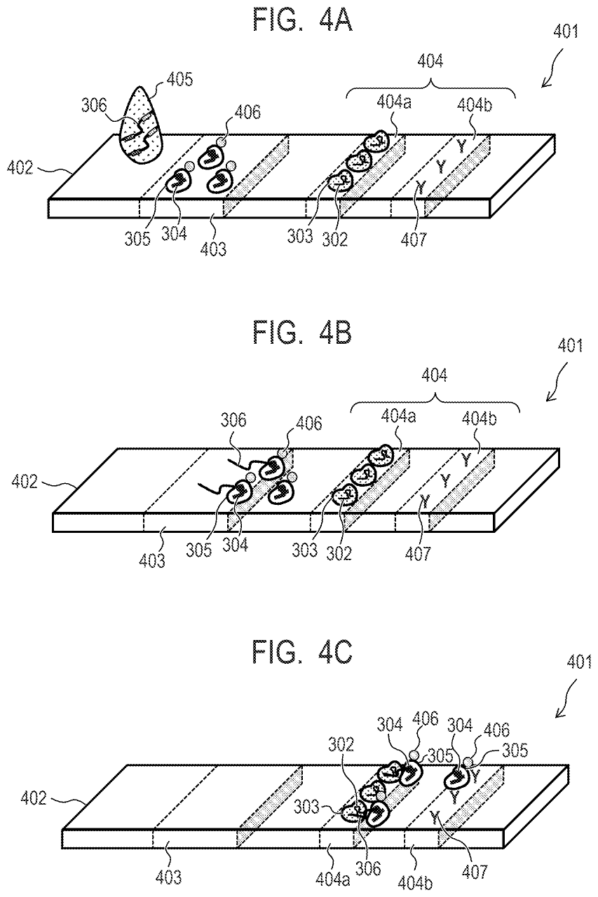

FIGS. 4A to 4C are schematic diagrams illustrating detection of a target nucleic acid using a lateral flow assay.

A lateral flow strip 401 for use in a lateral flow assay includes a sample pad 402, a conjugation pad 403, and a reaction membrane 404. In the case where the specimen containing the target nucleic acid is reacted with a reagent containing the second guide RNA and the second Cas protein in advance, the conjugation pad 403 may be omitted. Further, a waste liquid reservoir or the like for recovering the liquid after passing through the reaction membrane 404 may be provided.

A second guide RNA 304 and a second Cas protein 305 are added to the conjugation pad 403 in advance. The reaction membrane 404 comprises a test line 404a and a control line 404b separated from each other by a fixed distance. In the test line 404a, a complex of the first guide RNA 302 and the first Cas protein 303 is immobilized in advance. In the control line 404b, an anti-dCas9 antibody 407 is immobilized as a substance for complementing the second guide RNA 304 and the second Cas protein 305 complex in advance.

As shown in FIG. 4A, a specimen 405 containing a target nucleic acid 306 is first added to a sample pad 402 on a lateral flow strip 401. The specimen 405 is moved from the sample pad 402 to the condensation pad 403 by capillary action. Thus, the second guide RNA 304 and the second Cas protein 305 bind to the second base sequence of the target nucleic acid 306.

The second guide RNA 304 and the second Cas protein 305 are preferably reacted beforehand to form a complex. Gold nanoparticles 406 are immobilized as a labeling substance on the second Cas protein 305.

It should be noted that a known technique can be used as a method for immobilizing gold nanoparticles on a Cas protein. For example, there are passive adsorption methods and covalent bonding methods. For example, when the covalent bonding method is used, it is possible to bond a Cas protein to the terminal carboxy group on the surface of a gold nanoparticle by using a carboxy group-modified gold nanoparticle and using a carbodiimide crosslinking agent (EDC). When EDC is added, it is also possible to form a stable amide bond by adding sulfo-NHS.

The specimen 405 comprising the target nucleic acid 306 moves toward the reaction membrane 404 as shown in FIG. 4B. The second guide RNA 304 and the second Cas protein 305 bound to the target nucleic acid 306 are then captured at test line 404a as shown in FIG. 4C.

Specifically, in test line 404a, a complex of first guide RNA 302 and first Cas protein 303 binds to the first base sequence of target nucleic acid 306. This forms a complex comprising target nucleic acid 306, first guide RNA 302, first Cas protein 303, second guide RNA 304 and second Cas protein 305 on test line 404a.

Unreacted second guide RNA 304 and second Cas protein 305 pass through test line 404a and flow to control line 404b where they bind to and are captured by anti-dCas9 antibody 407 as shown in FIG. 4C.

Detection of the complex captured in test line 404a and the unreacted second guide RNA 304 and second Cas protein 305 is performed using gold nanoparticles 406, respectively, bound to the second Cas protein. In this example, gold nanoparticles are used as a labeling substance, but silver nanoparticles, biotin, fluorescein, or the like also can be used as a labeling substance instead. The detection method of the labeling substance may be appropriately selected according to the type of the labeling substance.

When the specimen 405 comprises the target nucleic acid 306, two lines of the test line 404a and the control line 404b are detected. On the other hand, if the specimen 405 does not contain the target nucleic acid 306 and contains a DNA of HPV 18 that is highly homologous to the target nucleic acid but has a different sequence, it results as follows.

That is, since the second guide RNA 304 is highly specific as described above, first in the sample pad 402, the second guide RNA 304 and the second Cas protein 305 do not bind to the DNA of the HPV 18.

The specimen 405 then moves toward the reaction membrane 404, but essentially the first guide RNA 302 and the first Cas protein 303 do not bind to the DNA of HPV 18. Thus, both the complex comprising the second guide RNA 304 and the second Cas protein 305 and the HPV 18 DNA pass through the test line 404a.

Subsequently, the second Cas protein 305 binds to the anti-dCas9 antibody 407 immobilized on the control line 404b. Thus, a complex comprising the second guide RNA 304 and the second Cas protein 305 is captured at the control line 404b. As a result, no line of the test line 404a is detected, but only one line of the control line 404b is detected.

Since the specificity of the first guide RNA 302 is somewhat low as described above, the DNA of the HPV 18 may non-specifically bind to the first guide RNA 302 and the first Cas protein 303 in the test line 404a. That is, off-target in the CRISPR-Cas system may occur.

Again, however, because of the high specificity of the second guide RNA 304, the nucleic acid of HPV 18 does not bind the second guide RNA to the second Cas protein, as described above. That is, the complex containing the DNA of the HPV 18 immobilized on the test line 404a does not have a labeling substance such as gold nanoparticles. Therefore, even when the first guide RNA 302 and the first Cas protein 303 misdetect the DNA of the HPV 18, the line of the test line 404a is not detected. As described above, HPV 16 can be detected separately from HPV 18 by using the detection method of the target nucleic acid according to the present invention.

### Example 3

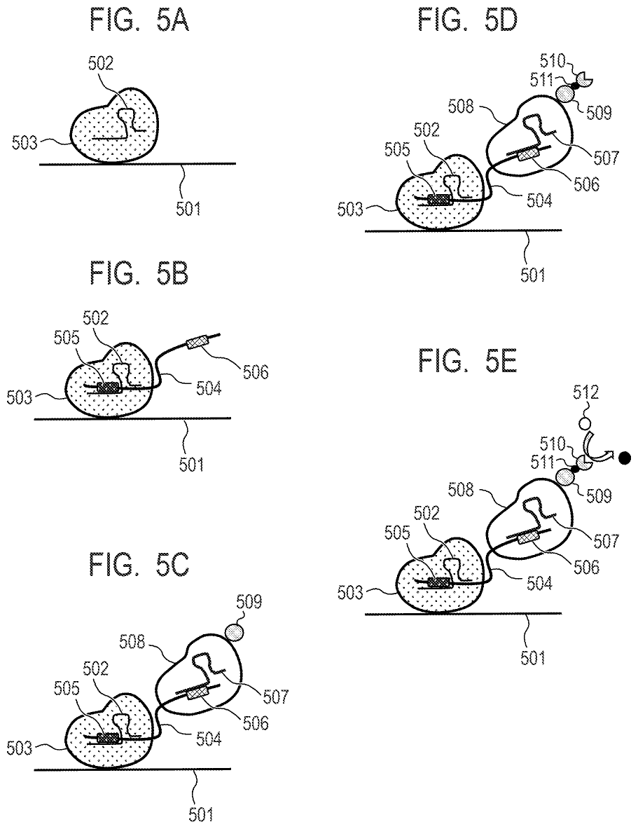

In this example, the first Cas protein is immobilized on a substrate and the target nucleic acid is detected. The steps of the detection method according to the present embodiment are shown in FIGS. 5A to 5E.

Double-Stranded DNA was used as the target nucleic acid, and dCas9 of NEW ENGLAND BIOLABS was used as the first Cas protein and the second Cas protein.

The sequence of one strand, the first base sequence and the second base sequence of the double-stranded DNA used as the target nucleic acid are shown below. Both the first base sequence and the second base sequence are the sequences in the following strands. The sequences of the first and second guide RNA are also shown below.

Sequence of one strand of double-stranded DNA
used as target nucleic acid
(SEQ ID NO: 7)
TCGAAGGGTGATTGGATCGGAGATAGGATGGGTCAATCGTAGGG
ACAATCGAAGCCAGAATGCAAGGGTCAATGGTACGCAGAATGGATGGCAC
TTAGCTAGCCAGTTAGGATCCGACTATCCAAGCGTGTATCGTACGGTGTA
TGCTTCGGAGTAACGATCGCACTAAGCATGGCTCAATCCTAGGCTGATAG
GTTCGCACATAGCATGCCACATACGATCCGTGATTGCTAGCGTGATTCGT
ACCGAGAACTCACGCCTTATGACTGCCCTTATGTCACCGCTTATGTCTCC
CGATATCACACCCGTTATCTCAGCCCTAATCTCTGCGGTTTAGTCTGGCC
TTAATCCATGCCTCATAGCTACCCTCATACCATCGCTCATACCTTCCGAC
ATTGCATCCGTCATTCCAACCCTGATTCCTACGGTCTAACCTAGCCTCTA
TCCTACCCAGTTAGGTTGCCTCTTAGCATCCCTGTTACGTACGCTCTTAC
CATGCGTCTTACCTTGGCACTATCGATGGG
First base sequence
(SEQ ID NO: 8)
AGGGTCAATGGTACGCAGAA
Second base sequence
(SEQ ID NO: 9)
CATTCCAACCCTGATTCCTA
First guide RNA
(SEQ ID NO: 10)
AGGGUCAAUGGUACGCAGAAGUUUUAGAGCUAGAAAUAGCAA
GUUAAAAUAAGGCUAGUCCGUUAUCAACUUGAAAAAGUGGCACCGAGUC
GGUGCUUUU
Second guide RNA
(SEQ ID NO: 11)
CAUUCCAACCCUGAUUCCUAGUUUUAGAGCUAGAAAUAGCAA
GUUAAAAUAAGGCUAGUCCGUUAUCAACUUGAAAAAGUGGCACCGAGUC
GGUGCUUUU

The flow of detection of the target nucleic acid in this embodiment will be described with reference to FIGS. 5A to 5E.

First, as shown in FIG. 5A, a complex comprising the first guide RNA 502 and the first Cas protein 503 was immobilized on the substrate 501. Specifically, a solution containing the first guide RNA 502 at a concentration of 25 μM and a solution containing the first Cas protein 503 at a concentration of 20 μM were prepared and mixed in a volume ratio of 1:1. The reaction was then allowed to proceed at 37° C. for 30 minutes to form a first guide RNA 502-first Cas protein 503 complex.

The first Cas protein 503 was immobilized on the substrate 501 by physical adsorption. An Immulon 2HB plate was used as the substrate 501. The first guide RNA 502-first Cas protein 503 complex was diluted with PBS so that the concentration of first Cas protein 503 was 10 μg/mL, and 100 μL of the resulting solution was added to substrate 501. Thereafter, by leaving the substrate 501 overnight at 4° C., the first guide RNA 502 and the first Cas protein 503 were immobilized on the substrate 501.

After immobilization, the liquid on the substrate 501 was removed, followed by cleaning the substrate 501 with PBS containing 0.5% Tween 20 (Hereinafter referred to as PBST). BSA was then added to the substrate 501 for blocking and washed with PBST.

Subsequently, as shown in FIG. 5B, a target nucleic acid 504 having a first base sequence 505 and a second base sequence 506 was added, and the first guide RNA 502 and the first Cas protein 503 were bonded to the first base sequence 505. The target nucleic acid 504 was prepared to have a concentration of 0 nM to 5 nM, and 100 μL of each solution was added to the substrate 501 and reacted at 37° C. for 2 hours.

Next, a complex comprising a second guide RNA 507 and a second Cas protein 508 to which a labeling substance is bound was prepared. The dCas9 used in this example was tagged in advance, and the tag was used to bind biotin 509 to the second Cas protein 508 according to the protocol using SNAP-biotin from NEW ENGLAND Co.

Thereafter, a solution containing the second guide RNA 507 at a concentration of 2.5 μM and a solution containing the second Cas protein 508 conjugated with biotin 509 at a concentration of 2 μM were mixed at a volume ratio of 1:1. The resulting mixed solution was then reacted at 37° C. for 30 minutes to produce a second guide RNA 507-second Cas protein 508 complex. After the reaction, in order to remove unreacted SNAP-biotin, ultrafiltration was performed, and the solution was diluted so that the concentration of the second Cas protein was 50 nM. PBST containing 0.5% BSA was used for dilution.

The thus prepared second guide RNA 507-second Cas protein 508 complex was added to the substrate 501 and bound to the second base sequence 506 of the target nucleic acid 504. Thus, as shown in FIG. 5C, a complex comprising the target nucleic acid 504, the first guide RNA 502, the first Cas protein 503, the second guide RNA 507 and the second Cas protein 508 was immobilized on the substrate 501. The reaction for binding the second guide RNA 507-second Cas protein 507 complex to the second base sequence 506 of the target nucleic acid 504 was carried out at 37° C. for 2 hours.

Thereafter, the liquid on the substrate 501 was removed, the substrate 501 was washed with PBS, and as shown in FIG. 5D, avidin 511 labeled with horseradish peroxidase (HRP) 510 was added and reacted at room temperature for 2 hours. Thereafter, the liquid on the substrate 501 was removed and the substrate 501 was cleaned with PBST.

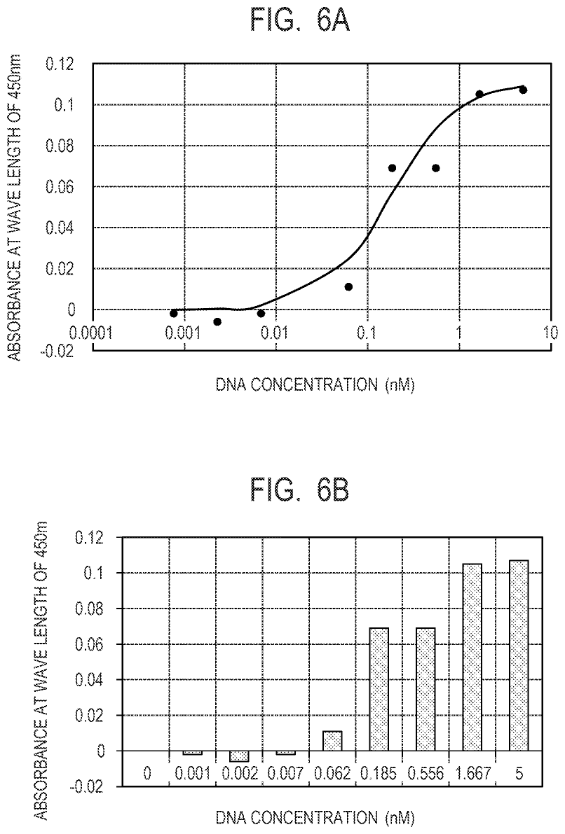

Subsequently, as shown in FIG. 5E, the HRP 510 and the coloring substrate 512 were reacted with each other for about 10 minutes using a peroxidase coloring kit, followed by the addition of a stop solution, and the absorbance at a wavelength of 450 nm was measured. The result of the absorbance measurement is shown in FIGS. 6A and 6B.

FIG. 6A is a graph showing the relationship between the concentration of the target nucleic acid and the absorbance obtained by the measurement, and FIG. 6B is a scatter plot showing the relationship between the concentration of the target nucleic acid and the absorbance obtained by the measurement. As shown in FIGS. 6A and 6B, the change in absorbance depending on the concentration of the target nucleic acid 504 can be observed, and it has been demonstrated that the target nucleic acid 504 can be detected. Further, as shown in FIG. 6B, by preparing a calibration curve from the data obtained for the target nucleic acid 504 and the absorbance, it was demonstrated that the concentration of the target nucleic acid 504 can be measured using the detection method according to the present invention.

According to the present invention, there can be provided a detection method for a target nucleic acid that is rapid, versatile and has higher specificity, and a kit for carrying out the detection method for the target nucleic acid.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2020-154144, filed Sep. 14, 2020, which is hereby incorporated by reference herein in its entirety.

What is claimed is:

1. A detection method for a target nucleic acid comprising:
a first step of contacting a target nucleic acid with a first guide RNA and a first Cas protein, the first guide RNA recognizing a first base sequence of the target nucleic acid;
a second step of contacting the target nucleic acid with a second guide RNA and a second Cas protein, the second guide RNA recognizing a second base sequence of the target nucleic acid and the second base sequence being different from the first base sequence; and
a third step of detecting a complex comprising the target nucleic acid, the first guide RNA, the first Cas protein, the second guide RNA, and the second Cas protein, wherein, in the complex, the first guide RNA and the first Cas protein are bound to the first base sequence, and the second guide RNA and the second Cas protein are bound to the second base sequence.

2. The detection method for a target nucleic acid according to claim 1, wherein the target nucleic acid is a double-stranded DNA, and the first Cas protein and the second Cas protein each are nuclease-deficient Cas9 protein.

3. The detection method for a target nucleic acid according to claim 1, wherein, in the first step, the target nucleic acid is contacted with the first guide RNA and the first Cas protein after mixing the first guide RNA and the first Cas protein.

4. The detection method for a target nucleic acid according to claim 1, wherein, in the second step, the target nucleic acid is contacted with the second guide RNA and the second Cas protein after mixing the second guide RNA and the second Cas protein.

5. The detection method for a target nucleic acid according to claim 1, wherein the target nucleic acid has 20 or more bases between the first base sequence and the second base sequence.

6. The detection method for a target nucleic acid according to claim 1, wherein the target nucleic acid has 100 or more bases between the first base sequence and the second base sequence.

7. The detection method for a target nucleic acid according to claim 1, wherein the target nucleic acid has 200 or more bases between the first base sequence and the second base sequence.

8. The detection method for a target nucleic acid according to claim 1, wherein the second guide RNA or the second Cas protein is bound to a second labeling substance, and in the third step, the complex is detected by a second signal generated by using the second labeling substance.

9. The detection method for a target nucleic acid according to claim 8, wherein the second signal is generated by using at leaset one selected from a group consisting of a radioactive substance, an enzyme, a capture molecule, a fluorescent substance, a luminescent substance, a metal particle, a protein-protein binding pair, and a protein-antibody binding pair.

10. The detection method for a target nucleic acid according to claim 8, wherein the first guide RNA or the first Cas protein is bound to a first labeling substance, the first labeling substance is different from the second labeling substance, and in the third step, the complex is detected by two ways of a detection by a first signal generated using the first labeling substance and a detection by the second signal.

11. The detection method for a target nucleic acid according to claim 8, wherein the third step includes a step of obtaining information on a concentration of the target nucleic acid based on the second signal.

12. The detection method for a target nucleic acid according to claim 11, wherein the information on the concentration of the target nucleic acid includes at least one selected from a group consisting of presence or absence of the target nucleic acid, a value of the concentration of the target nucleic acid, and a ratio of the concentration of the target nucleic acid to a reference value of the concentration of the target nucleic acid.

13. The detection method for a target nucleic acid according to claim 1, wherein in the third step, the complex is detected by applying a lateral flow assay.

14. The detection method for a target nucleic acid according to claim 1, wherein in the third step, the complex is detected by utilizing aggregation of a latex particle.

15. A kit for detecting a target nucleic acid, the kit comprising a first guide RNA, a first Cas protein, a second guide RNA, and a second Cas protein, wherein
the first guide RNA has a sequence capable of recognizing a first base sequence of the target nucleic acid,
the second guide RNA has a sequence capable of recognizing a second base sequence of the target nucleic acid, and
the second base sequence is different from the first base sequence.

16. The kit according to claim 15, wherein the first Cas protein and the second Cas protein each are nuclease-deficient Cas9 protein.

17. The kit according to claim 15, wherein the second guide RNA or the second Cas protein is bound to a second labeling substance.

18. The kit according to claim 17, wherein the second labeling substance is a substance used for generation of a second signal generated by using at leaset one selected from a group consisting of a radioactive substance, an enzyme, a capture molecule, a fluorescent substance, a luminescent substance, a metal particle, a protein-protein binding pair, and a protein-antibody binding pair.

19. The kit according to claim 15 further comprising a lateral flow strip.

20. The kit according to claim 15, further comprising a latex particle.
